(19) United States
(12) Patent Application Publication
(10) Pub. No.: US 2022/0338980 A1
(43) Pub. Date: Oct. 27, 2022

(54) PROSTHETIC VALVE WITH ASYMMETRIC LEAFLETS

(71) Applicant: W.L. Gore & Associates, Inc., Newark, DE (US)

(72) Inventors: Kyle W. Colavito, Flagstaff, AZ (US); Michael J. Shepard, Flagstaff, AZ (US)

(21) Appl. No.: 17/632,948

(22) PCT Filed: Aug. 10, 2020
(86) PCT No.: PCT/US20/45614

§ 371 Date: Feb. 4, 2022

Related U.S. Application Data
(60) Provisional application No. 62/884,559, filed on Aug. 8, 2019.

(51) Int. Cl. A61F 2/24

(52) U.S. Cl. CPC A61F 2/2412; A61F 2210/0076; A61F 2230/008; A61F 2250/0014

(57) ABSTRACT

Described embodiments are directed toward prosthetic valves having leaflets that move asymmetrically in that in the fully open position, the leaflet first side region opens less than the leaflet second side region. Asymmetric opening and final open position, in synchrony with the other leaflets having similar motion and final open position creates spiral flow exiting the open valve that may, among other things, increases blood flow on the downstream side of the leaflet and thus reduces stagnation of the blood that might lead to thrombus formation. Controlled asymmetric movement of the leaflet reduces closing volume by initiating closure on the leaflet first side region and finishing closures on the leaflet second side region.

### FIELD

The present disclosure relates generally to prosthetic valves and more specifically, prosthetic valve devices and methods with asymmetric flow characteristics.

### BACKGROUND

Bioprosthetic valves have been developed that attempt to mimic the function and performance of a native valve. Flexible leaflets are fabricated from biological tissue or synthetic materials. In some valve designs the flexible leaflets are coupled onto a relatively rigid frame that supports the leaflets and provides dimensional stability when implanted. The leaflets move under the influence of fluid pressure. In operation, the leaflets open when the upstream fluid pressure exceeds the downstream fluid pressure and close when the downstream fluid pressure exceeds the upstream fluid pressure. The free edges of the leaflets coapt under the influence of downstream fluid pressure closing the valve to prevent downstream blood from flowing retrograde through the valve.

The flexible nature of the leaflet can create regions of blood stagnation behind the leaflet when in the open position potentially causing blood clots to form at the leaflet base and near the attachment of the leaflet to the support structure.

What is needed in the art is a flexible leaflet prosthetic valve that provides a more controlled leaflet movement that reduces closing volume and potential for blood stagnation near the leaflet and near any attachment of the leaflet to a support structure.

### SUMMARY

In accordance with an embodiment (1), a prosthetic valve comprises a leaflet frame defining a plurality of leaflet windows each defining an attachment zone, each attachment zone defining three sides of an asymmetric acute trapezoid, and a leaflet extending from each of the leaflet windows, the leaflet defining a leaflet base and a free edge opposite the leaflet base and two leaflet sides having dissimilar length, the leaflet base and two leaflet sides being coupled to the respective attachment zone, wherein adjacent leaflet free edges are configured to coapt in a progressive manor when closing and disengage in a progressive manor when opening.

In accordance with an embodiment (2), a prosthetic valve comprises a tubular support structure defining a lumen, and a plurality of leaflets coupled to the support structure, each leaflet defining a leaflet base and a free edge opposite the leaflet base and two leaflet sides having dissimilar length, the leaflet base and two leaflet sides being coupled to the support structure and in cooperative engagement with respective leaflets such that adjacent free edges coapt when in a closed position so as to occlude the lumen, wherein adjacent leaflet free edges are configured to coapt in a progressive manor from adjacent the support structure to a point of convergence when closing and disengage in a progressive manor from the point of convergence to the support structure when opening.

In accordance with an embodiment (3), a prosthetic valve comprises a leaflet frame defining a plurality of leaflet windows each defining an attachment zone in the shape of an asymmetric parabola, and a leaflet extending from each of the leaflet windows, the leaflet defining two leaflet sides having dissimilar length, the two leaflet sides being coupled to the attachment zone, wherein adjacent leaflet free edges are configured to coapt in a progressive manor when closing and disengage in a progressive manor when opening.

In accordance with an embodiment (4), a prosthetic valve comprises a tubular support structure defining a lumen, and a plurality of leaflets coupled to the support structure, each leaflet defining two leaflet sides having dissimilar length, the leaflet base and two leaflet sides in the shape of an asymmetric parabola being coupled to the support structure and in cooperative engagement with respective leaflets such that adjacent free edges coapt when in a closed position so as to occlude the lumen, wherein adjacent leaflet free edges are configured to coapt in a progressive manor from adjacent the support structure to a point of convergence when closing and disengage in a progressive manor from the point of convergence to the support structure when opening.

In accordance with an embodiment (5), a prosthetic valve comprises a plurality of leaflets, each leaflet defines a leaflet first side region and a leaflet second side region opposite from the leaflet first side region, each leaflet defining a leaflet base and a leaflet free edge opposite from the leaflet base, each leaflet first side region is coupled with the leaflet second side region of an adjacent leaflet at a commissure, the leaflet base of each of the plurality of leaflets together define a lumen, at least one of the leaflet second side regions extend further into the lumen than the leaflet first side region when the leaflets are in a fully open position.

In accordance with an embodiment (6), a prosthetic valve comprises a leaflet frame defining a plurality of leaflet windows each defining an attachment zone in the shape of a symmetric parabola and a first commissure post and second commissure post extending therefrom, and a leaflet extending from each of the leaflet windows, each leaflet having a first tab attachment line operable to couple with the first commissure post, and a second tab attachment line operable to couple with the second commissure post, the second tab attachment line has a greater angle relative to a valve longitudinal axis as compared with the first tab attachment line having a smaller angle.

In accordance with an embodiment (7), a prosthetic valve comprises a leaflet frame defining a plurality of leaflet windows each defining an attachment zone in the shape of a symmetric parabola and a first commissure post and second commissure post extending therefrom, and a leaflet extending from each of the leaflet windows, each leaflet having a first tab attachment line operable to couple with the first commissure post, and a second tab attachment line operable to couple with the second commissure post, the first tab attachment line has a greater length relative to the second tab attachment line.

In accordance with an embodiment (8), a prosthetic valve of any of the embodiments 1 through 7, wherein when in an open position, the leaflet second side region contributes to a smaller geometric orifice area as compared with the leaflet first side region.

In accordance with an embodiment (9), a prosthetic valve of embodiment 8, wherein the leaflet first side region opens further than the leaflet second side region during forward flow.

In accordance with an embodiment (10), a prosthetic valve of embodiment 8 wherein when in the open position, the leaflet second side region has between a 10 to 70 percent smaller geometric orifice area as compared with the leaflet first side region.

In accordance with an embodiment (11), a prosthetic valve of any of the embodiments 1 through 7, wherein the leaflet first side region has a first surface area and the leaflet second side region has a second surface area, the first surface area being greater than the second surface area.

In accordance with an embodiment (12), a prosthetic valve of any of the embodiments 1 through 7, wherein the leaflet comprises at least one layer of a composite material.

In accordance with an embodiment (13), a prosthetic valve of any of the embodiments 1 through 7 wherein the attachment line of the leaflet first side region is longer than the attachment line of the leaflet second side region.

In accordance with an embodiment (14), a prosthetic valve of any of the embodiments 1 through 7, wherein the leaflet comprises a polymeric material.

In accordance with an embodiment (15), a prosthetic valve of any of the embodiments 1 through 7, wherein each of the leaflet windows includes a leaflet window first side, a leaflet window second side opposite the leaflet window first side, and a leaflet window base therebetween, wherein a leaflet window side of one leaflet window is interconnected with a leaflet window side of an adjacent leaflet window by the leaflet window base, wherein the leaflet first side region being coupled to the leaflet window first side, the leaflet second side region being coupled to the leaflet window second side and the leaflet base being coupled to the leaflet window base.

In accordance with an embodiment (16), a prosthetic valve of any of the embodiments 3 through 4, wherein each of the leaflet windows includes a leaflet window first side and a leaflet window second side opposite the leaflet window first side, wherein a leaflet window side of one leaflet window is interconnected with a leaflet window side of an adjacent leaflet window, wherein the leaflet first side region being coupled to the leaflet window first side, and the leaflet second side region being coupled to the leaflet window second side.

In accordance with an embodiment (17), a prosthetic valve of any of the embodiments 15 and 16, wherein two adjacent leaflet window first side and leaflet window second side terminates at a commissure post.

In accordance with an embodiment (18), a prosthetic valve of any of embodiment 17, each leaflet including a free edge, a base opposite from the free edge and coupled to the leaflet window base, wherein the leaflet reinforcing member extends to the free edge of the leaflet.

In accordance with an embodiment (19), a prosthetic valve of embodiment 17, further comprising a vertical element extending from each of the commissure posts.

In accordance with an embodiment (20), a prosthetic valve of any of the embodiments 1 through 7, wherein the leaflet comprises a polymeric material.

In accordance with an embodiment (21), a prosthetic valve of embodiment 20, wherein the leaflet comprises a laminate.

In accordance with an embodiment (22), a prosthetic valve of embodiment 21, wherein the laminate has more than one layer of a fluoropolymer membrane.

In accordance with an embodiment (23), a prosthetic valve of embodiment 20, wherein the leaflet comprises at least one fluoropolymer membrane having a plurality of pores and an elastomer present in substantially all of the pores of the at least one fluoropolymer membrane.

In accordance with an embodiment (24), a prosthetic valve of embodiment 23, wherein the film comprises less than 80% fluoropolymer membrane by weight.

In accordance with an embodiment (25), a prosthetic valve of embodiment 23, wherein the elastomer comprises (per)fluoroalkylvinylethers (PAVE).

In accordance with an embodiment (26), a prosthetic valve of embodiment 23, wherein the elastomer comprises a copolymer of tetrafluoroethylene and perfluoromethyl vinyl ether.

In accordance with an embodiment (27), a prosthetic valve of embodiment 23, wherein the fluoropolymer membrane comprises ePTFE.

In accordance with an embodiment (28), a method of treating a failing or dysfunctional native heart valve with a prosthetic valve, the method comprising replacing the native valve with a prosthetic valve in accordance with any one of embodiments 1 through 27.

Describe embodiments are directed to flexible leaflet prosthetic valves in which the leaflets move into the open and closed position in a controlled manner. A leaflet is a flexible membrane that is coupled to a support structure about an attachment line. Opposite from the attachment line is the leaflet free edge. Under changing fluid pressure the leaflet is free to move between an open and closed position pivoting substantially about the leaflet attachment line with the leaflet free edge moving within the valve orifice. By way of reference in discussing the difference between a symmetric and asymmetric leaflet, a symmetric leaflet may have an attachment line in the shape of a symmetric parabola, for example, which is symmetric about an axis of symmetry through the vertex. The leaflet may be defined as having a leaflet first side and a leaflet second side on either side of the axis of symmetry, also referred herein as a dividing line. In the case where the leaflet is symmetric, the leaflet first side and leaflet second side will move substantially symmetrically between the open and closed position; in other words, the leaflet will move as a mirror image along the dividing line. In the case where the leaflet is asymmetric about the dividing line, as in an asymmetric parabola where the curvature of one side is not the same as the curvature of the second side, the leaflet first side will move differently from that of the leaflet second side in response to changing fluid pressure.

In accordance with embodiments, each leaflet moves asymmetrically, and further, when in the fully open position, a leaflet first side region opens to a greater extent than a leaflet second side region. Such asymmetric opening position, in synchrony with the other leaflets having the same opening position, creates a spiral flow pattern to the fluid exiting the open valve that assists in creating an axial vortex flow that increases blood flow on the downstream side of the leaflet. When in the open position, the leaflet second side region extends further into the valve lumen than the leaflet first side which exposes the leaflet downstream side of the second side region to retrograde blood flow which increases washout of the blood from the leaflet downstream side. This increase in blood flow on the downstream side of the leaflet reduces the potential for stagnation of the blood that might lead to thrombus formation.

Further, in the closing phase where the blood flow is reversed, the leaflet second side region which extends into the valve lumen to a greater extent than the leaflet first side region, the leaflet second side region responds to the reversed flow and moves toward the closed position before the leaflet first side region. This closing dynamic may reduce the stress on the leaflet as it flexes about the attachment line or throughout the leaflet as it bends between the open and close position. Further, controlled asymmetric movement of the leaflet may reduce closing volume by initiating closure of the leaflet second side region before the leaflet second side region, reducing leaflet resistance to closure by, in part, allowing one region of the leaflet to close before another region.

Described embodiments are directed to flexible leaflet prosthetic valves in which the frame attachment edge onto which the leaflet attachment edge is coupled, and thus, the leaflet, is not symmetrical about an axis that axially bisects the leaflet, so as to provide asymmetric opening and closing of the leaflet, in accordance with an embodiment. In accordance with some embodiments, the leaflet, as axially dissected, is defined as having a first side region having a geometry defining a smaller surface area relative to a second side region. The difference in surface area provides that the leaflet moves into the open and closed position in a preferential manner, with one side region having a different response to changing fluid pressure relative to the other side region. Further, the difference in surface area positions the open leaflet in the lumen of the valve in an asymmetric configuration so as to provide an increased blood flow behind the leaflet and, in particular, where the leaflet attaches to the leaflet frame.

In accordance with an embodiment, a prosthetic valve comprises a leaflet frame and a plurality of leaflets coupled to the leaflet frame. Each leaflet has a free edge, a leaflet first side, a leaflet second side, and a leaflet base therebetween. The leaflet first side, leaflet second side, and leaflet base are coupled to the leaflet frame. Each leaflet has a leaflet first side region adjacent the leaflet first side, a leaflet second side region adjacent the leaflet second side. The leaflet first side region has a surface area that is less than the surface area of the leaflet second side region.

In accordance with another embodiment, a prosthetic valve comprises a frame having a generally tubular shape with attached film. The frame defines a plurality of leaflet windows. Each leaflet window defines a leaflet window first side, a leaflet window second side, and a leaflet window base. The leaflet window first side and the leaflet window second side diverge from the leaflet window base. A film defines at least one leaflet extending from each of the leaflet windows. Each leaflet has a free edge, a leaflet first side that is coupled to the leaflet window first side, a leaflet second side that is coupled to the leaflet window second side, and a leaflet base therebetween that is coupled to the leaflet window base. Each leaflet has a leaflet first side region adjacent the leaflet first side and extending to a substantially axial line from the leaflet free edge to the intersection between the leaflet window first side and the leaflet window base, a leaflet second region adjacent the leaflet second side and extending to a substantially axial line from the leaflet free edge to the intersection between the leaflet window second side and the leaflet window base. The leaflet first side region has a surface area that is less than the surface area of the leaflet second region.

In accordance with another embodiment, a prosthetic valve comprises a plurality of leaflets where each leaflet includes a leaflet first side and a leaflet second side opposite from the leaflet first side. Each leaflet first side is coupled with the leaflet second side of an adjacent leaflet at a commissure. The plurality of leaflets defines a lumen when the leaflets are in an open position. Each of the leaflet second side regions extend further into the lumen than each of the leaflet first side regions when the leaflets are in the open position.

### BRIEF DESCRIPTION OF THE DRAWINGS

The accompanying drawings are included to provide a further understanding of the present disclosure and are incorporated in and constitute a part of this specification, illustrate embodiments described herein, and together with the description serve to explain the principles discussed in this disclosure.

FIG. 1A is a perspective view of a prosthetic valve in accordance with known art;

FIG. 1B is an axial perspective view of the embodiment of the valve of FIG. 1A;

FIG. 1C is a side view of the leaflet frame of the prosthetic valve of FIGS. 1A-1B, wherein the leaflet frame has been longitudinally cut and laid open;

FIG. 2A is a side view of a prosthetic valve having been bisected and unrolled to a flat orientation to more clearly see the elements, in accordance with an embodiment;

FIG. 2B is an embodiment of a leaflet suitable to couple with a frame of the prosthetic valve of FIG. 2A;

FIG. 3A is an axial view of the prosthetic valve in accordance with the embodiment of FIG. 2A in a closed position;

FIG. 3B is an axial view of the prosthetic valve in accordance with the embodiment of FIG. 2A in a partially open position;

FIG. 3C is an axial view of the prosthetic valve in accordance with the embodiment of FIG. 2A in an open position;

FIG. 4 is an embodiment of a leaflet;

FIG. 5A is a side view of a prosthetic valve, in accordance with an embodiment’

FIG. 5B is a perspective view of the prosthetic valve of FIG. 5B;

FIG. 5C is an embodiment of the prosthetic valve of FIG. 5A having been bisected and unrolled to a flat orientation to more clearly see the elements;

FIG. 6A is an axial view of the prosthetic valve in accordance with the embodiment of FIG. 5A in a closed position;

FIG. 6B is an axial view of the prosthetic valve in accordance with the embodiment of FIG. 5A in a partially open position; and

FIG. 6C is an axial view of the prosthetic valve in accordance with the embodiment of FIG. 5A in an open position.

### DETAILED DESCRIPTION

Persons skilled in the art will readily appreciate that various aspects of the present disclosure can be realized by any number of methods and apparatus configured to perform the intended functions. Stated differently, other methods and apparatuses can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Although the embodiments herein may be described in connection with various principles and beliefs, the described embodiments should not be bound by theory. For example, embodiments are described herein in connection with prosthetic valves, more specifically cardiac prosthetic valves. However, embodiments within the scope of this disclosure can be applied toward any valve or mechanism of similar structure and/or function. Furthermore, embodiments within the scope of this disclosure can be applied in non-cardiac applications.

The term leaflet as used herein in the context of prosthetic valves is a component of a one-way valve wherein the leaflet is operable to move between an open and closed position under the influence of a pressure differential. In an open position, the leaflet allows blood to flow through the valve. In a closed position, the leaflet substantially blocks retrograde flow through the valve. In embodiments comprising multiple leaflets, each leaflet cooperates with at least one neighboring leaflet to block the retrograde flow of blood. The pressure differential in the blood is caused, for example, by the contraction of a ventricle or atrium of the heart, such pressure differential typically resulting from a fluid pressure building up on one side of the leaflets when closed. As the pressure on an inflow side of the valve rises above the pressure on the outflow side of the valve, the leaflets opens and blood flows therethrough. As blood flows through the valve into a neighboring chamber or blood vessel, the pressure on the inflow side equalizes with the pressure on the outflow side. As the pressure on the outflow side of the valve rises above the blood pressure on the inflow side of the valve, the leaflet returns to the closed position generally preventing retrograde flow of blood through the valve.

The term membrane as used herein refers to a sheet of material comprising a single composition, such as, but not limited to, expanded fluoropolymer.

The term composite material as used herein refers to a combination of a membrane, such as, but not limited to, expanded fluoropolymer, and an elastomer, such as, but not limited to, a fluoroelastomer. The elastomer may be imbibed within a porous structure of the membrane, coated on one or both sides of the membrane, or a combination of coated on and imbibed within the membrane.

The term laminate as used herein refers to multiple layers of membrane, composite material, or other materials, such as elastomer, and combinations thereof.

The term film as used herein generically refers to one or more of the membrane, composite material, or laminate.

The term biocompatible material as used herein generically refers to a film or a biological material, such as, but not limited to, bovine pericardium.

The term leaflet window is defined as that space that a frame defines from which a leaflet is coupled and extends therefrom. The leaflet may extend from frame elements or adjacent to frame elements and spaced apart therefrom. The frame elements define a “window” into which the leaflet extends.

As used herein, “couple” means to join, connect, attach, adhere, affix, or bond, whether directly or indirectly, and whether permanently or temporarily.

Embodiments herein include various apparatus, systems, and methods for a prosthetic valve suitable for surgical and transcatheter placement, such as, but not limited to, cardiac valve replacement. The valve is operable as a one-way valve wherein the valve defines a valve lumen into which leaflets open to permit flow and close so as to occlude the valve lumen and prevent flow in response to differential fluid pressure.

Described embodiments are directed to flexible leaflet prosthetic valves in which the leaflets move into the open and closed position in a more controlled manner. The leaflets move in synchrony with each other. In described embodiments, each leaflet moves asymmetrically in that a leaflet second side region of the leaflet moves toward the closed position before a leaflet first side region, in contrast with a leaflet that moves symmetrically where the leaflet first side region moves in the same way as the leaflet second side region. Further, in the fully open position, the leaflet second side opens less than the leaflet first side, in contrast with a leaflet that moves symmetrically where the leaflet first side region fully opens to the same extent as the leaflet second side region. The leaflet first side region of one leaflet is adjacent to the leaflet second side region of an adjacent leaflet. Such asymmetric closing and final open position, in synchrony with the other leaflets having the same closing and final open position, creates a spiral flow pattern to the fluid exiting the open valve that assists in creating an axial vortex flow that increases blood flow on the downstream side of the leaflet and thus reduces the potential for stagnation of the blood that might lead to thrombus formation. In contrast, a symmetric leaflet movement and open position, the fluid flow through the valve will not have a spiral flow characteristic. Further, controlled asymmetric movement of the leaflet may reduce closing volume by initiating closure of the leaflet second side region and finishing with closure of the leaflet first side region, reducing leaflet buckling resistance to closure by, in part, allowing one side region of the leaflet to close before another side region which leads to a more rolling larger radius bending dynamics rather than a tight smaller radius bending dynamics. Further, the leaflet open position is controlled such that the leaflet second side region extends further into the valve lumen of the valve relative to the leaflet first side region to further expose the leaflet downstream side to the retrograde blood flow dynamics which increases washout of the blood from the leaflet downstream side and exposes the leaflet downstream side to improved reverse blood flow which may assist in leaflet closing during the closing phase.

In accordance with embodiments provided herein, at least a portion of the leaflet second side region is configured to be more resistant to motion as compared with the leaflet first side region. The resistant to motion may be affected in a number of ways, including, but not limited to, providing a difference in leaflet surface area between the leaflet first side region as compared with the leaflet second side region, configuring the geometry of the leaflet frame to which the leaflet is attached such that the bending characteristics of the leaflet first side region is different as compared with the leaflet second side region, and combinations thereof.

Embodiments provided herein address controlled leaflet opening and closing. Embodiments provided herein provide a feature of different leaflet geometry of one side region of the leaflet relative to the other side region. The asymmetric geometry of the leaflet is operable to initiate leaflet closing of one side region before the other side region of the leaflet. Therefore, the leaflet will open asymmetrically with respect to the leaflet free edge rather than symmetrically as with a leaflet having a uniform or symmetric leaflet geometry. This asymmetric movement has the potential to minimize crease formation of the leaflet, which is of particular importance in thin, high-modulus leaflets. If the leaflet bending is uncontrolled, not only may creases form in the leaflet, but crease intersections may lead to formation of compound wrinkles that oppose bending and slow down or disrupt the motion of the leaflet, both in opening and closing. Embodiments provided herein control the dynamics of the leaflet motion in a controlled asymmetric opening and closing of the leaflet so as to minimize crease formation.

Embodiments provided herein address blood pooling or stagnation that can lead to clot formation behind the leaflet and along the intersection of the leaflet and the frame when the leaflet is open. Embodiments provided herein provide a feature of differing leaflet geometry from one side region of the leaflet to the other side region. A leaflet second side region of the leaflet is configured to open to a lesser extent than a leaflet first side region. Since the second side region of the leaflet does not open fully and therefore protrudes into the flow more so than the first side region, retrograde blood flow may better extend behind the leaflet, the downstream side, producing a washing effect along the attachment of the leaflet to the frame and, in particular, at the base of the leaflet on the downstream side of the leaflet. Since the second side region of the leaflet protrudes into the retrograde flow more so than the first side region, when the flow reverses, the second side region protruding into the flow will actuate the closing of the valve much sooner and in a more controlled manner. Therefore, the leaflet will close asymmetrically from the second side region to the first side region with respect to the leaflet free edge rather than randomly and chaotically as with a very thin and flexible leaflet having a uniform or symmetric stiffness property. This asymmetric movement minimizes crease formation and provides a faster closing response, which is of particular importance in thin, high-modulus leaflets. Embodiments provided herein control leaflet open configuration and dynamics that may provide increased flow behind the leaflet to increase washing and reduce blood stagnation, minimization of crease formation, and a faster closing response provided by the controlled asymmetric closing of the leaflet. The asymmetric opening may also ensure a more uniform opening over a variety of loading conditions. This may be advantageous in lower flow conditions by ensuring that all leaflets open in a more consistent manner.

In accordance with embodiments presented below, the asymmetric shape of the leaflet is at least in part determined by the shape of the leaflet window, to which the leaflet is coupled. The frame elements that define the leaflet window define an asymmetric geometry about a line of axis that is parallel with the axis of the valve. In accordance with embodiments provided below, various asymmetric leaflet window geometries are presented that are predetermined to control the leaflet dynamics as provided. By way of consistency, in the various embodiments provided below, the leaflet window frame elements define a longer leaflet attachment line on a first side which is to the left of a dividing axis as for those frame elements on a second side to the right of the dividing axis. It is understood that this is for convenience of discussion and not limited thereto.

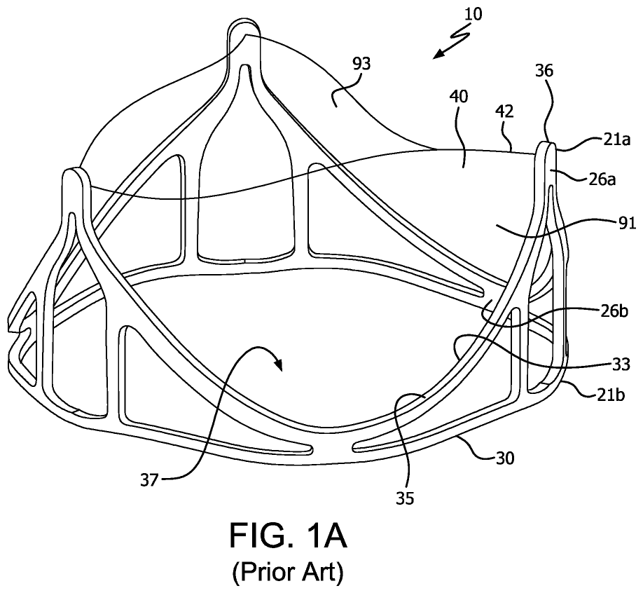

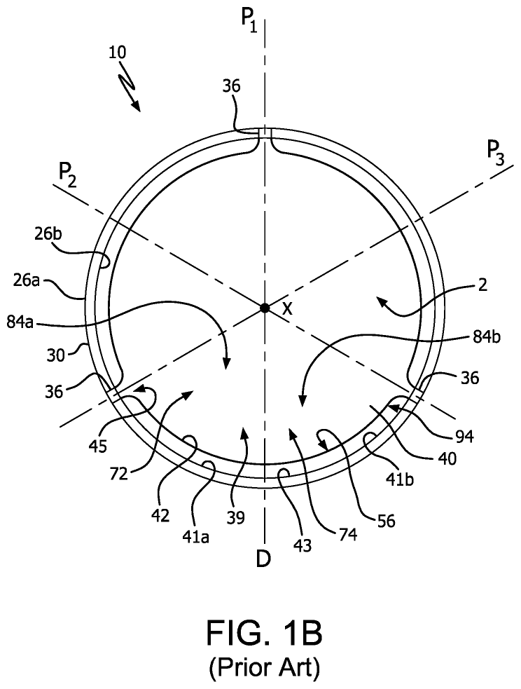

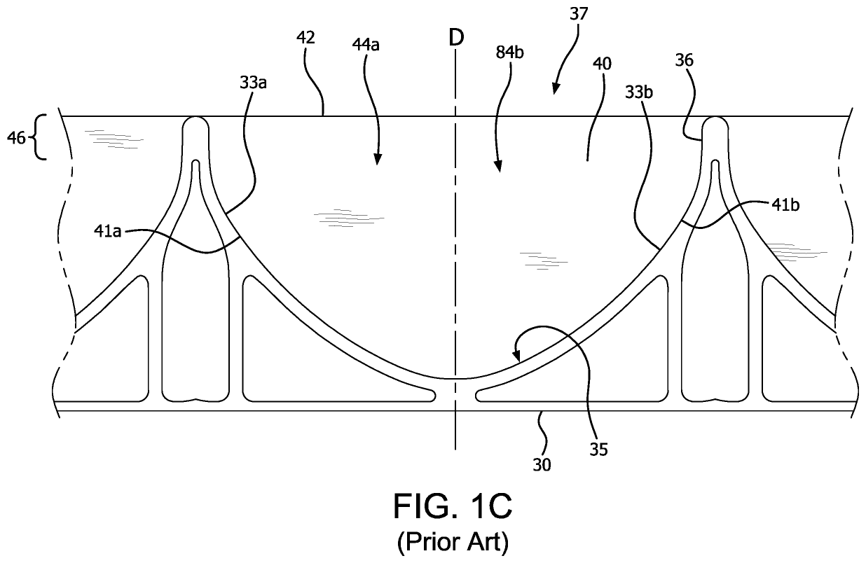

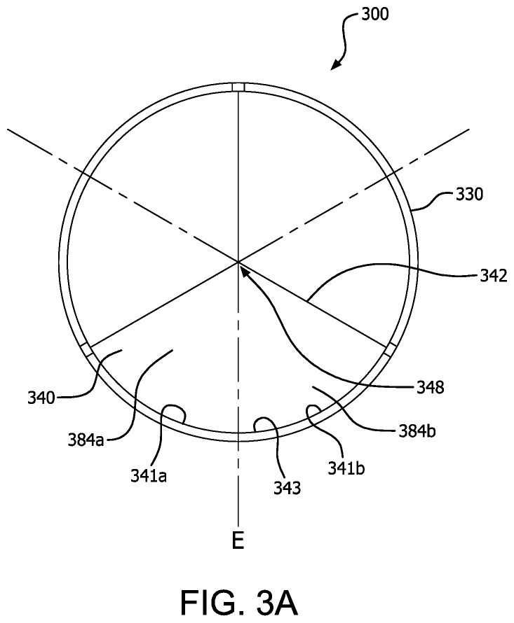

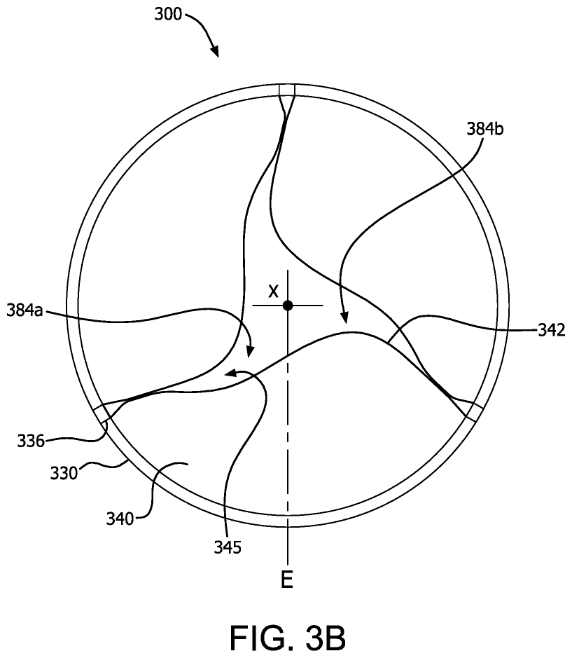

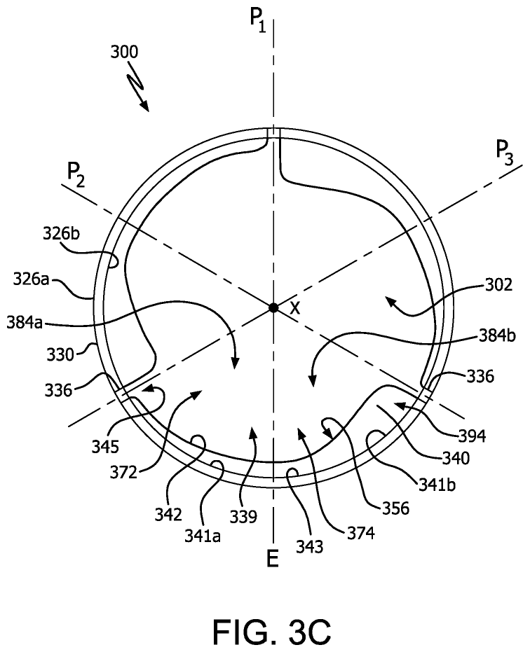

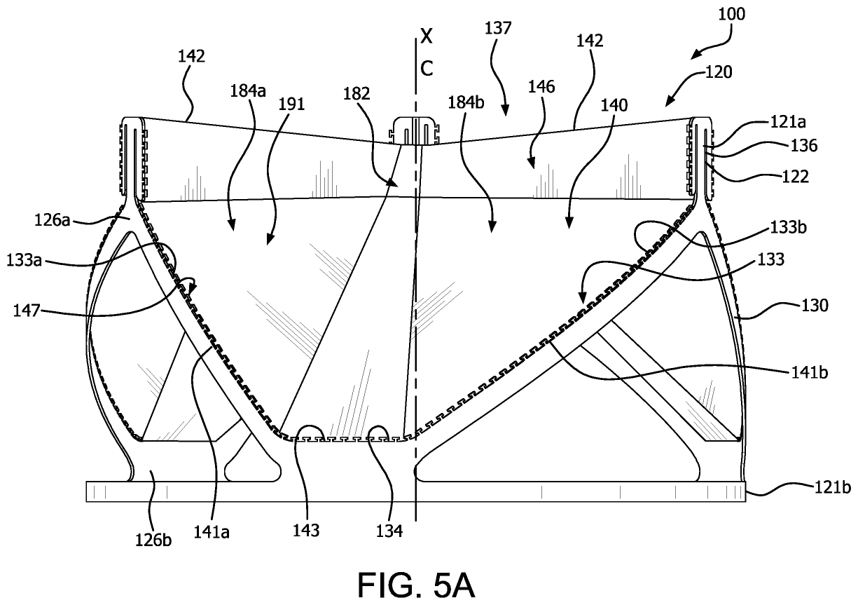

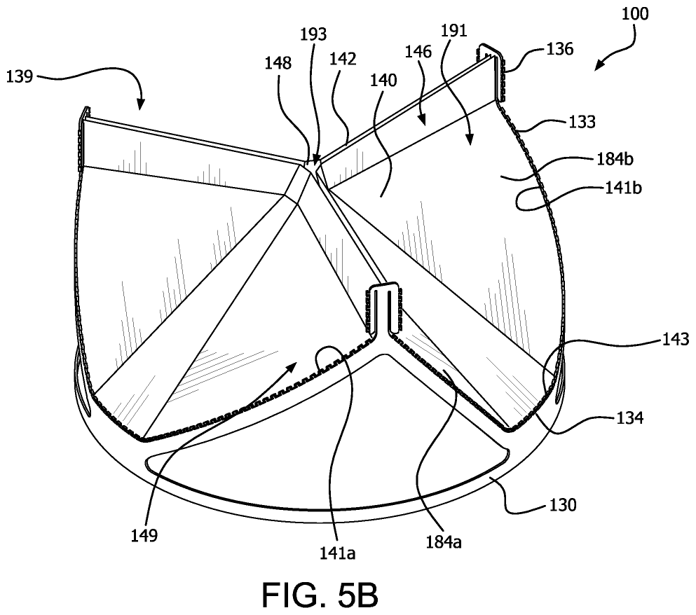

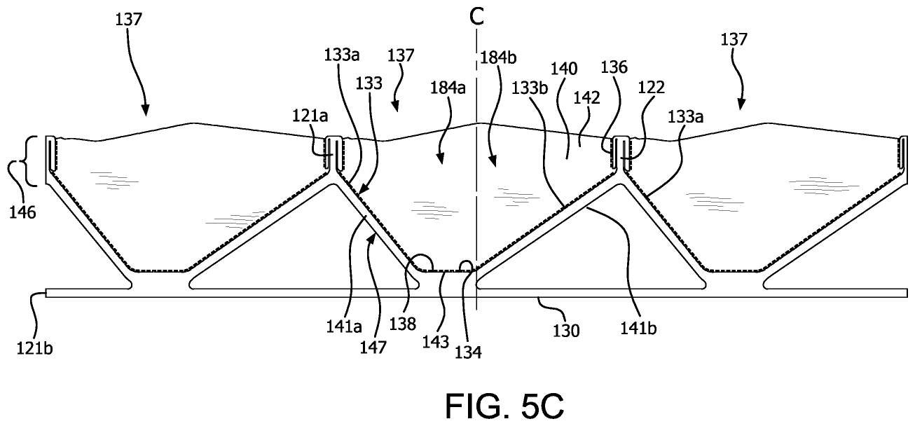

Asymmetry in the leaflet window of the leaflet frame maybe be affected by various geometries. In the embodiment of FIG. 1A, the leaflet window 137 defines the shape of three sides of an asymmetric trapezoid. The embodiment of FIG. 3A is an example of a leaflet window 237 that is in the shape of an asymmetric parabola. The embodiment of FIG. 5 is another example of a leaflet window 337 in the shape of an asymmetric parabola with an asymmetric commissure post attachment which may also result in an asymmetric coaptation region which may affect leaflet dynamics in the proscribed way.

Symmetric Valve

FIG. 1A is a perspective view of a prosthetic valve 10, in accordance with the prior art, which is used herein as a frame of reference as an example of a valve with leaflets 40 that are geometrically symmetric and move symmetrically. FIG. 1B is an axial view of the prosthetic valve 10 of FIG. 1A in an open position showing that the leaflets 40 are uniformly and symmetrically open. The prosthetic valve 10 comprises a leaflet frame 30 and leaflets 40 that are attached to the leaflet frame 30. In FIG. 1A, the leaflets 40 are shown mostly open but it is understood that when prosthetic valve 10 is fully closed the leaflet free edges 42 of the leaflets 40 will come together to coapt under the influence of downstream fluid pressure which results in closing the prosthetic valve 10 to prevent downstream fluid from flowing retrograde through the prosthetic valve 10.

Symmetric Frame

Referring to FIGS. 1A-1C, the leaflet frame 30 is a generally tubular member, in accordance with the prior art, which is used herein as a frame of reference as an example of a prosthetic valve with leaflets 40 that are geometrically symmetric and move symmetrically. The leaflet frame 30 comprises a frame first end 21a and a frame second end 21b opposite the frame first end 21a. The leaflet frame 30 comprises a frame outer surface 26a and a frame inner surface 26b opposite the frame outer surface 26a, as shown in FIG. 1A. The leaflet frame 30 defines commissure posts 36 that couple to the leaflet free edges 42. The commissure posts 36 are defined by a vertical element 22.

FIG. 10 is a side view of the leaflet frame 30 of the prosthetic valve 10 of FIGS. 1A-1B, wherein the leaflet frame 30 has been longitudinally cut and laid open to better illustrate the elements of the generally tubular-shaped leaflet frame 30, in accordance with the prior art, which is used herein as a frame of reference as an example of a valve with leaflets 40 that are geometrically symmetric and move symmetrically. In FIG. 10, a leaflet window 37 is defined by a window first side 33a and a window second side 33b, which are defined by a dividing line D which is parallel with the valve axis X in FIG. 1B. It should be noted that in accordance with the prior art, the window first side 33a is symmetric with the window second side 33b, and in this example, is in the shape of a parabola. The dividing line D is located along a line of symmetry passing through the apex of the parabola. This example is used as a frame of reference to compare with the embodiments of an asymmetric valves provided herein.

In FIG. 1B, a leaflet 40 is shown coupled to and located within the window 37. The leaflet 40 is divided in half by dividing line D into a leaflet first side region 184a adjacent to the window first side 133a and the leaflet second side region 184b adjacent to the window second side 133b.

Asymmetric Valve

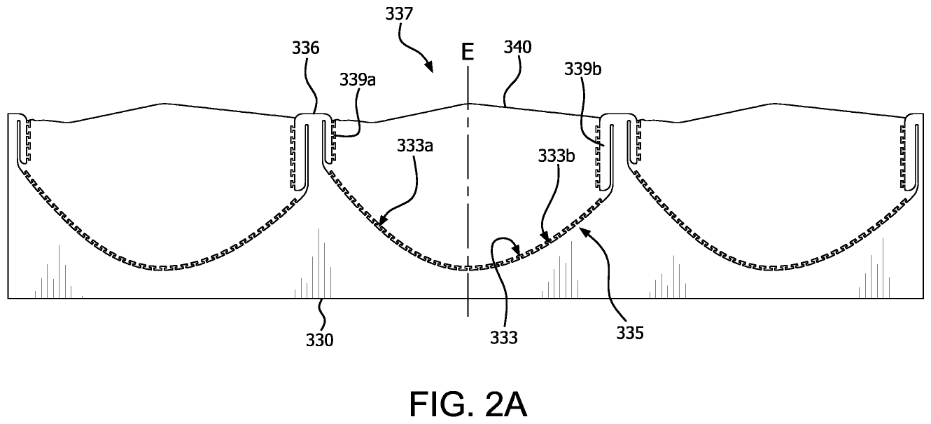

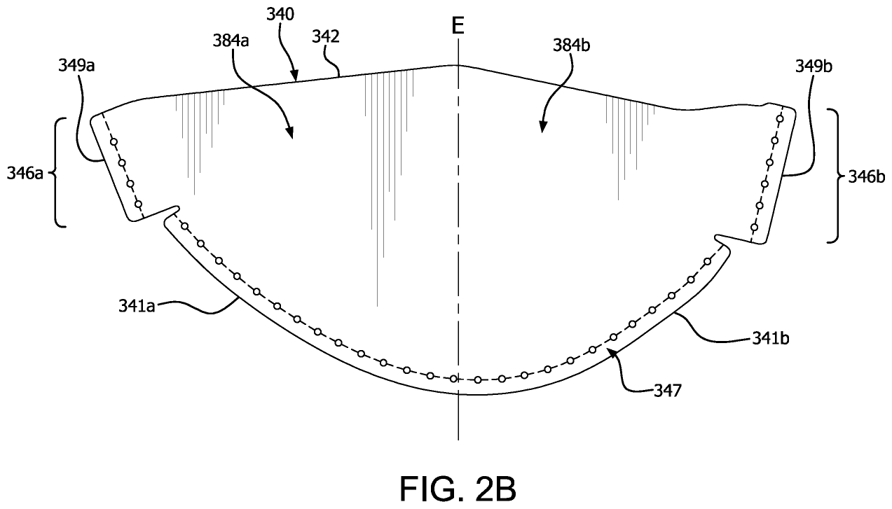

FIG. 2A is a side view of the leaflet frame 330 of a prosthetic valve 100 of FIGS. 3A-3C, wherein the leaflet frame 330 has been longitudinally cut and laid open to better illustrate the elements of the generally tubular-shaped leaflet frame 330, in accordance with an embodiment. The leaflet frame 330 comprises a plurality of leaflet frame elements 335 defining leaflet windows 337. In the embodiment of FIG. 2A three interconnected leaflet windows 337 are defined, where a window first side 333a of one leaflet window 337 is interconnected with an adjacent leaflet window second side 333b of an adjacent leaflet window 337 by a commissure post 336 therebetween.

In this embodiment the shape of the leaflet window 337 is that of an asymmetric parabola with an asymmetric commissure post 336. The leaflet frame 330 defines a plurality of interconnected asymmetric parabolic leaflet frame elements 335 terminating at commissure posts 336 defining leaflet windows 337. Each parabolic leaflet frame element 335 may be defined by a window first side 333a and window second side 333b on either side of a dividing line E which bisects the asymmetric parabolic leaflet frame element 335 passing through the vertex. The dividing line E is parallel with the axis X of the valve 300, similarly as shown in FIG. 3C.

In this embodiment, the window first side 333a defines half of a first parabola and the window second side 333b defines half of a second parabola that has a greater slope than the first parabola. Although the window first side 333a and window second side 333b are defined as parabolas having different slopes in accordance with an embodiment, embodiments also include other geometric shapes of the window first side 333a and window second side 333b that are different from each other. In accordance with another embodiment provided below, by way of example, the window first side 333a and window second side 333b have a combined shape of three sides of an asymmetric trapezoid.

Referring again to FIG. 2A, the commissure posts 336 extend from an intersection of intersecting parabolic leaflet frame elements 335. The commissure post 336 includes a first commissure post attachment element 339a and a second commissure post attachment element 339b. The length of the first commissure post attachment element 339a is longer than the length of the second commissure post attachment element 339b, to accommodate, in part, the different slopes of the window first side 333a and window second side 333b. The different

FIG. 2B is an embodiment of a leaflet 340 suitable to couple with the leaflet window 337 of the leaflet frame 320 of FIG. 2A. The leaflet 340 has a complimentary shape of the asymmetric parabola of the window first side 333a and the window second side 333b, with an attachment line 347 that is operable to couple with the window first side 333a and the window second side 333b. A dividing line E that is parallel with the axis X of the prosthetic valve 300 bisects the leaflet 340 defining a first leaflet side region 384a operable to couple with the window first side 133a, and a second leaflet side region 384b operable to couple with the leaflet window second side 133b. Further, the leaflet first side region 384a includes a first commissure tab 349a adjacent the leaflet free edge 340 operable to couple with the first commissure post attachment element 339a. The leaflet second side region 384b includes a second commissure tab 349b adjacent the leaflet free edge 340 operable to couple with the second commissure post attachment element 339b. The first commissure tab 349a corresponds to a first coaptation region 346a and the second commissure tab 349b corresponds to a second coaptation region 346b, resulting in a coaptation region that may vary in height from the first commissure tab 349a to the second commissure tab 349b.

The leaflet frame 330 may comprise a cut tube, or any other element suitable for the particular purpose. The leaflet frame 330 may be etched, cut, laser cut, or stamped into a tube or a sheet of material, with the sheet then formed into a tubular or substantially cylindrical structure, among other methods and means.

The leaflet frame 330 can comprise any metallic or polymeric material that is biocompatible. For example, the leaflet frame 330 can comprise a material, such as, but not limited to nitinol, cobalt-nickel alloy, stainless steel, or polypropylene, acetyl homopolymer, acetyl copolymer, ePTFE, other alloys or polymers, or any other biocompatible material having adequate physical and mechanical properties to function as described herein.

FIG. 3A is an axial view of the prosthetic valve 300 when the prosthetic valve 300 is closed. Generally, about half of each leaflet free edge 342 abuts an adjacent half of a leaflet free edge 342 of an adjacent leaflet 340. The three leaflets 340 of the embodiment of FIG. 3A meet at a triple point 348, for the trileaflet valve. The valve lumen 302 is occluded when the leaflets 340 are in the closed position stopping fluid flow. Although the leaflet first side region 384a has a different geometry than the leaflet second side region 384b, the flexibility of the leaflets 340 allow for coaptation with the leaflet first side region 384a with an adjacent second side region 384b of an adjacent leaflet 340 allowing for proper closing of the prosthetic valve 300.

FIG. 3B is an axial view of the valve 300 in the partially open position or a partially closed position. The leaflet first side region 384a of one leaflet 340 is adjacent to the leaflet second side region 384b of an adjacent leaflet 340. The leaflet first side region 384a has a different geometry as compared to the leaflet second side region 384b. FIG. 3B illustrates the dynamics of the leaflet 340 in that the leaflet first side region 384a and the leaflet second side region 384b respond differently to the flow dynamics. As the leaflet 340 opens, the leaflet first side region 384a opens first and farther than the leaflet second side region 384b. The leaflet second side region 384b is operable to open less fully and may close more quickly as compared to the leaflet first side region 384a. This controlled motion provides a consistent leaflet motion from cycle to cycle imparting the benefits described herein, including, but not limited to, having a more controlled bending characteristic preventing leaflet creases, that is not random which may be found in a symmetric leaflet.

FIG. 3C is an axial view of the prosthetic valve 300 in the fully open position. As shown in FIG. 3C, the leaflets 340 do not completely open so as to not conform to the leaflet frame inner surface 326b. The leaflet frame inner surface 326b in cross-section transverse to the X axis defines a frame lumen 339 having a frame orifice area that is circular in shape. The leaflet first side region 384a opens more fully than the leaflet second side region 384b due to the asymmetric shape of the leaflet 340. It has been found that for this embodiment, a leaflet first side region 384a having a leaflet attachment line 347 defining a half-parabola with a lower slope will open more fully than the leaflet second side region 384b having a leaflet attachment line 347 defining a half-parabola having a great slope. This asymmetric opening of the prosthetic valve 300 creates a spiral downstream flow dynamic with the corresponding benefits as provided herein, such as, but not limited to, better fluid flow, known as wash-out, behind the leaflet 340 so as to prevent low flow or stagnant flow regions that may lead to thrombus formation in blood.

As the leaflet 340 cycles between the open and closed positions, the leaflets 340 generally flex about the leaflet attachment line 347. Since the leaflet first side region 384a has a different geometry than the leaflet second side region 384b, the leaflet second side 341b is operable to not flex or bend as much about the leaflet window second side 333a as compared with the leaflet first side 341a which defines a channel 345 between the leaflet first side 341a of one leaflet 340 and the leaflet second side 341b of an adjacent leaflet 340 when the leaflet 340 is not in the closed position, as shown in FIGS. 3B-3C. The channel 345 is defined when the leaflet 340 moves from the closed position. The channel 345 allows for fluid flow therethrough throughout the open phase of the leaflet 340 and thus reduces the potential for blood pooling, stagnation and clot formation between adjacent leaflets 340.

Since the leaflet 340 is asymmetric about the dividing line E, the leaflet second side region 384b does not open fully leaving a pocket 194 defined in part by the leaflet downstream side 191 adjacent the leaflet second side region 384b. As the blood exits the prosthetic valve 100, retrograde flow may enter the pocket 194 so as to wash out the area defined by the leaflet downstream side 191, preventing fluid stagnation and potential thrombus formation.

Referring again to FIG. 3C, by way of example, the axial view of the valve 300 shown is bisected into six segments by three planes P1, P2, P3 where each plane passes through one commissure post 336, the axis X and bisects the leaflet 340, defining a first segment 372 and a second segment 374. The portion of the leaflet in the second segment 374 defines a smaller geometric orifice area (GOA) than the portion of the leaflet in the first segment 372, by virtue of the leaflet second side region 384b extending further into the frame lumen 339 defined by the leaflet frame inner surface 326b. As such, the fluid dynamics through the first segment 372 will be different than that of the second segment 374 which will tend to impart a spiral component to the downstream flow field.

The leaflet 340 can be configured to actuate at a pressure differential in the blood caused, for example, by the contraction of a ventricle or atrium of the heart, such pressure differential typically resulting from a fluid pressure building up on one side of the valve 300 when closed. As the pressure on an inflow side of the valve 300 rises above the pressure on the outflow side of the valve 300, the leaflet 340 opens and blood flows therethrough. As blood flows through the valve 300 into a neighboring chamber or blood vessel, the pressure equalizes. As the pressure on the outflow side of the valve 300 rises above the blood pressure on the inflow side of the valve 300, the leaflet 340 returns to the closed position generally preventing the retrograde flow of blood through the inflow side of the valve 300.

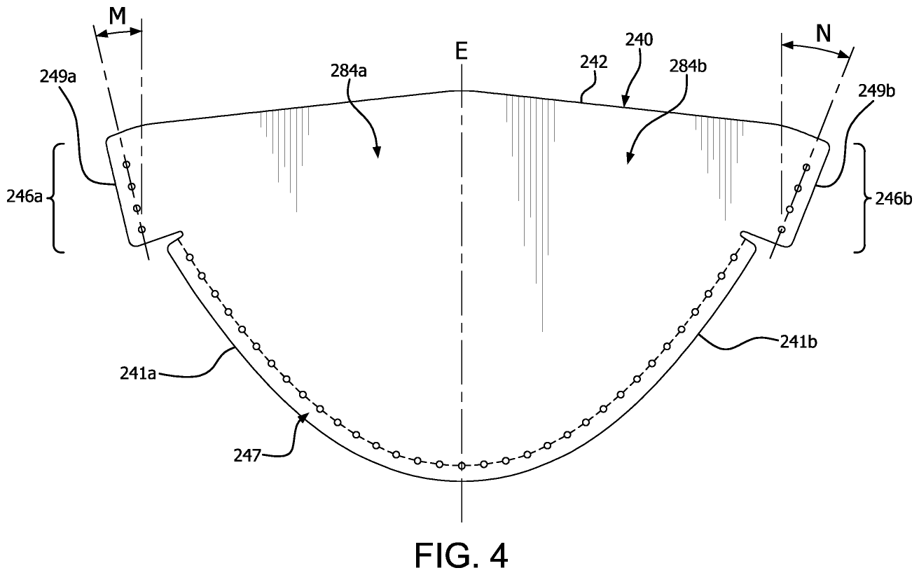

FIG. 4 is an embodiment of a leaflet 240 suitable to couple with a respectively shaped leaflet window of a leaflet frame, similar to the leaflet frame 320 of FIG. 2A, but matching the parabolic shape of the leaflet 240. In this embodiment, the leaflet 240 has an attachment line 247 defining a shape of a symmetric parabola. The leaflet first side region 384a includes a first commissure tab 249a adjacent the leaflet free edge 240, defined by dividing line F, operable to couple with a first commissure post attachment element of a leaflet frame about a first tab attachment line 245a. The leaflet second side region 284b includes a second commissure tab 249b adjacent the leaflet free edge 240 operable to couple with a second commissure post attachment element of a leaflet frame about a second tab attachment line 245b. In this embodiment, the length of the first tab attachment line 245a and the second tab attachment line 245b are the same. It has been found that a prosthetic valve with leaflets in accordance with this embodiment have asymmetric opening characteristics when the second tab attachment line 245b has a greater angle N relative to the valve longitudinal axis which is parallel with the dividing line F as compared with a first tab attachment line 245a of the first commissure tab 249b having a smaller angle M.

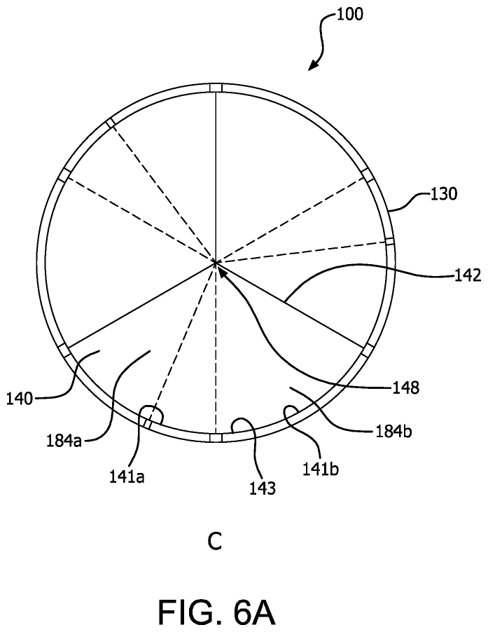

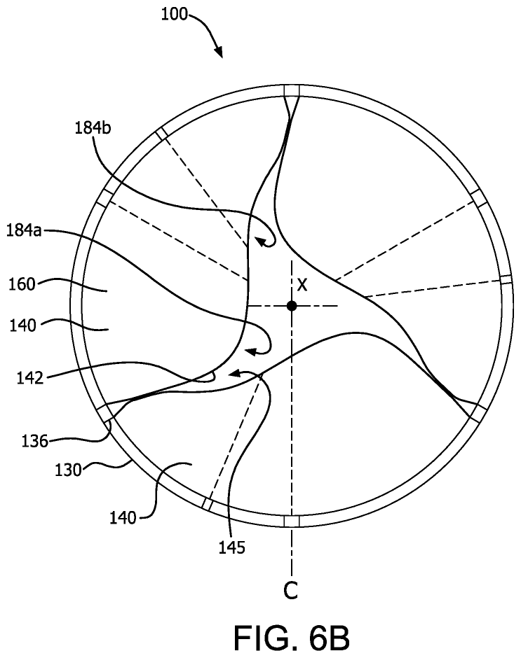

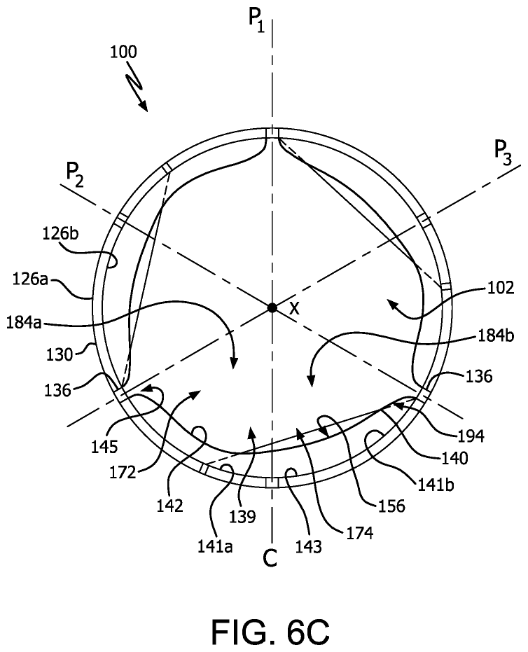

FIG. 5A is a side view of a prosthetic valve 100, in accordance with an embodiment. FIG. 5B is a perspective view of the prosthetic valve 100 of FIG. 1A. FIGS. 6A, 6B and 6C are axial views of the prosthetic valve 100 of FIG. 1A in a closed, partially open, and closed configuration, respectively. The prosthetic valve 100 comprises a leaflet frame 130 and leaflets 140 that are attached to the leaflet frame 130. In FIGS. 5A and 5B, the leaflets 140 are shown slightly open to better show the features but it is understood that the prosthetic valve 100 being fully closed will have the leaflet free edges 142 of the leaflets 140 coming together to coapt under the influence of downstream fluid pressure which results in closing the prosthetic valve 100 to prevent downstream blood from flowing retrograde through the prosthetic valve 100.

Referring to FIGS. 5A-5B and 6A-6C, the leaflet frame 130 is a generally tubular member, in accordance with an embodiment. The leaflet frame 130 comprises a leaflet frame first end 121a and a frame second end 121b opposite the leaflet frame first end 121a. The leaflet frame 130 comprises a leaflet frame outer surface 126a and a leaflet frame inner surface 126b opposite the leaflet frame outer surface 126a, as shown in FIG. 5A. The leaflet frame 130 defines commissure posts 136 that couple to the leaflet free edges 142. The commissure posts 136 are defined by a vertical element 122.

FIG. 5C is a side view of the leaflet frame 130 of the prosthetic valve 100 of FIGS. 5A-5B, wherein the leaflet frame 130 has been longitudinally cut and laid open to better illustrate the elements of the generally tubular-shaped leaflet frame 130, in accordance with an embodiment. In FIG. 5C, a leaflet window 137 is defined by a window first side 133a and a window second side 133b, interconnected by a window base 134 therebetween, which are defined by a dividing line C which is parallel with the valve axis X in FIG. 5A. It should be noted that with embodiments provided herein, the window first side 133a is not symmetric with the window second side 133b. The dividing line C is located in that position where a line of symmetry might be expected, halfway between two adjacent commissure posts 136. It is this asymmetry that provides the corresponding asymmetric dynamics of the leaflet 140 under fluid pressure. In the embodiment of FIG. 5C there are three interconnected leaflet windows 137, where a window first side 133a of one leaflet window 137 is interconnected with an adjacent window second side 133b of an adjacent leaflet window 137 by a commissure post 136 therebetween.

In FIG. 5C, a leaflet 140 is shown coupled to and located within the leaflet window 137. The leaflet is divided by dividing line C into a leaflet first side region 184a adjacent to the window first side 133a and the leaflet second side region 184b adjacent to the window second side 133b.

The leaflet frame 130 may comprise a cut tube, or any other element suitable for the particular purpose. The leaflet frame 130 may be etched, cut, laser cut, or stamped into a tube or a sheet of material, with the sheet then formed into a tubular or substantially cylindrical structure, among other methods and means.

The leaflet frame 130 can comprise any metallic or polymeric material that is biocompatible. For example, the leaflet frame 130 can comprise a material, such as, but not limited to nitinol, cobalt-nickel alloy, stainless steel, or polypropylene, acetyl homopolymer, acetyl copolymer, ePTFE, other alloys or polymers, or any other biocompatible material having adequate physical and mechanical properties to function as described herein

Referring to FIG. 5C, the leaflet frame comprises a plurality of spaced apart leaflet frame elements defining leaflet windows 137. Each of the leaflet windows 137 include a leaflet window first side 133a and leaflet window second side 133b interconnected by a base element 138 therebetween. In the embodiment of FIG. 2 there are three interconnected leaflet windows 137, where a leaflet window first side 133a of one leaflet window 137 is interconnected with an adjacent leaflet window second side 133b of an adjacent leaflet window 137 by a commissure post 136 therebetween.

Referring again to FIGS. 5A-5C, the leaflet frame first end 121a further comprises commissure posts 136 extending from an intersection of the leaflet frame elements defining the leaflet window first side 133a and leaflet window second side 133b of an adjacent leaflet window 137. The commissure post 136 may affect the leaflet free edge 142 so as to create a larger or wider coaptation region 146 where the adjacent leaflet free edges 142 come together when the prosthetic valve 100 is in the closed position.

In accordance with an embodiment of a prosthetic valve 100, each leaflet 140 has a leaflet first side 141a and a leaflet second side 141b, a leaflet base 143 and a leaflet free edge 142 opposite the leaflet base 143, wherein the leaflet first side 141a and a leaflet second side 141b diverge from the leaflet base 143, wherein the leaflet base 143 is substantially flat, as shown in FIG. 5C.

As the leaflets 140 cycle between the open and closed positions, the leaflets 140 generally flex about the leaflet base 143 and the portion of the leaflet window first side 133a and the leaflet window second side 133b to which the leaflets 140 are coupled. Since the leaflet first side region 184a has a different geometry than the leaflet second side region 184b, the leaflet second side 141b is operable to not flex or bend as much about the leaflet window second side 133b as compared with the leaflet first side 141a which defines a channel 145 between the leaflet first side 141a of one leaflet 140 and the leaflet second side 141b of an adjacent leaflet 140 when the leaflet is not in the closed position, as shown in FIGS. 6B and 6C. The channel 145 is defined when the leaflet 140 moves from the closed position. The channel 145 allows for fluid flow therethrough throughout the opening phase of the leaflet 140 and thus reduces the potential for blood pooling, stagnation and clot formation between the leaflet first side 141a of one leaflet and the leaflet second side 141b of an adjacent leaflet 140.

Since the leaflet 140 is asymmetric about the dividing line C, the leaflet second side region 184b does not open fully leaving a pocket 194 defined in part by the leaflet downstream side 391 adjacent the leaflet second side region 184b. As the blood exits the prosthetic valve 100, retrograde flow may enter the pocket 194 so as to wash out the area defined by the leaflet downstream side 191, preventing fluid stagnation and potential thrombus formation.

A geometric orifice area (GOA), as is known in the art, is an area measurement of an axial projection of an open area defined by the valve when in the fully open position. As explained below, a first portion of a leaflet will extend further into the valve lumen defined by the leaflet frame, that is, not open as much, than a second portion of the same leaflet, which opens further. From an axial viewpoint, the first portion of the leaflet will create a smaller GOA than the second portion of the leaflet.

Referring again to the embodiment of FIG. 5C, each leaflet window 137 is provided with a leaflet 140 which is coupled to a portion of the leaflet window side 133. Each leaflet 140 defines a leaflet free edge 142 and a leaflet base 143. The leaflet 140 is coupled to at least a portion of the leaflet window first side 133a, the leaflet window second side 133b and to the leaflet window base 134 about a leaflet attachment line 147. A dividing line C that is parallel with the axis X of the valve 100 bisects the leaflet window 137 and thus the leaflet 140 defining a first leaflet side region 184a including the leaflet window first side 133a and a portion of the leaflet window base 134 and a second leaflet side region 184b including the leaflet window second side 133b and a portion of the leaflet window base 134. The leaflet 140 has a leaflet upstream side 193 and a leaflet downstream side 191 opposite the leaflet upstream side 193, as shown in FIG. 5B. The leaflet upstream side 193 is that side of the leaflet 140 that faces away from the adjacent leaflet frame inner surface 126b of the leaflet frame 130 when in the open position and the leaflet downstream side 191 is that side of the leaflet 140 that faces toward the adjacent leaflet frame inner surface 126b of the leaflet frame 130 when in the open position, as shown in FIG. 6C.

When the leaflets 140 are in a fully open position, the prosthetic valve 100 presents a substantially circular valve lumen 102 as shown in FIG. 6C. Fluid flow is permitted through the valve lumen 102 when the leaflets 140 are in the open position. Since the leaflet 140 is asymmetric about the dividing line C, the leaflet second side region 184b does not open fully leaving a pocket 194 defined in part by the leaflet downstream side 191 adjacent the leaflet second side region 184b. As the blood exits the prosthetic valve 100, retrograde flow may enter the pocket 194 so as to wash out the area defined by the leaflet downstream side 191.

A geometric orifice area (GOA), as is known in the art, is an area measurement of an axial projection of an open area defined by the valve when in the fully open position. As explained below, a first portion of a leaflet will extend further into the valve lumen defined by the leaflet frame, that is, not open as much, than a second portion of the same leaflet, which opens further. From an axial viewpoint, the first portion of the leaflet will create a smaller GOA than the second portion of the leaflet.

FIG. 6C is an axial view of the prosthetic valve 100 in the fully open position. As shown in FIG. 6C, the leaflets 140 do not completely open so as to conform to the leaflet frame inner surface 126b, therefore projecting a smaller geometric orifice area compared with an orifice area of a frame without leaflets. The leaflet frame inner surface 126b in cross-section transverse to the X axis defines a frame lumen 139 having a frame orifice area that is circular in shape.

FIG. 6B is an axial view of the valve 300 in the partially open position or a partially closed position. The leaflet first side region 384a of one leaflet 340 is adjacent to the leaflet second side region 384b of an adjacent leaflet 340. The leaflet first side region 384a has a different geometry as compared to the leaflet second side region 384b.

FIG. 6B is an axial view of the valve 100 in the partially open position or a partially closed position. The leaflet first side region 184a of one leaflet 140 is adjacent to the leaflet second side region 184b of an adjacent leaflet 140. The leaflet first side region 184a has a different geometry as compared to the leaflet second side region 184b. FIG. 6B illustrates the dynamics of the leaflet 140 in that the leaflet first side region 184a and the leaflet second side region 184b respond differently to the flow dynamics. As the leaflet 140 opens, the leaflet first side region 184a opens first and farther than the leaflet second side region 184b. The leaflet second side region 184b is operable to open less fully and may close more quickly as compared to the leaflet first side region 184a. This controlled motion provides a consistent leaflet motion from cycle to cycle imparting the benefits described herein, including, but not limited to, having a more controlled bending characteristic preventing leaflet creases, that is not random which may be found in a symmetric leaflet.

As the leaflets 140 cycle between the open and closed positions, the leaflets 140 generally flex about the leaflet base 143 and the portion of the leaflet window first side 133a and the leaflet window second side 133b to which the leaflets 140 are coupled. Since the leaflet first side region 184a has a different geometry than the leaflet second side region 184b, the leaflet first side 141a is operable to not flex or bend as much about the leaflet window first side 133a as compared with the leaflet second side 141b which defines a channel 145 between the leaflet first side 141a of one leaflet 140 and the leaflet second side 141b of an adjacent leaflet 140 when the leaflet is not in the closed position, as shown in FIGS. 6B and 6C. The channel 145 is defined when the leaflet 140 moves from the closed position. The channel 145 allows for blood flow therethrough throughout the opening phase of the leaflet 140 and thus reduces the potential for blood pooling, stagnation and clot formation between the leaflet first side 141a and the leaflet window first side 133a, and the leaflet second side 141b and the leaflet window second side 133b, and therebetween.

When the prosthetic valve 100 is closed, generally about half of each leaflet free edge 142 abuts an adjacent half of a leaflet free edge 142 of an adjacent leaflet 140, as shown in FIG. 6A. The three leaflets 140 of the embodiment of FIG. 6A meet at a triple point 148, for the trileaflet valve. The valve lumen 102 is occluded when the leaflets 140 are in the closed position stopping fluid flow. Although the leaflet first side region 184a has a different geometry than the leaflet second side region 184b, the flexibility of the leaflets 140 allow for coaptation with the leaflet first side region 184a with an adjacent second side region 184b of an adjacent leaflet 140 allowing for proper closing of the valve 100.

Referring again to FIG. 6A, by way of example, the axial view of the prosthetic valve 100 shown is bisected into six segments by three planes P1, P2, P3 where each plane passes through one commissure post 136, the axis X and bisects the leaflet 140, defining a first segment 172 and a second segment 174. The portion of the leaflet in the second segment 174 defines a smaller geometric orifice area (GOA) than the portion of the leaflet in the first segment 172, by virtue of the leaflet second side region 184b extending further into the frame lumen 139 defined by the leaflet frame inner surface 126b. As such, the fluid dynamics through the first segment 172 will be different than that of the second segment 174 which will tend to impart a spiral component to the downstream flow field.

The leaflet 140 can be configured to actuate at a pressure differential in the blood caused, for example, by the contraction of a ventricle or atrium of the heart, such pressure differential typically resulting from a fluid pressure building up on one side of the valve 100 when closed. As the pressure on an inflow side of the valve 100 rises above the pressure on the outflow side of the valve 100, the leaflet 140 opens and blood flows therethrough. As blood flows through the valve 100 into a neighboring chamber or blood vessel, the pressure equalizes. As the pressure on the outflow side of the valve 100 rises above the blood pressure on the inflow side of the valve 100, the leaflet 140 returns to the closed position generally preventing the retrograde flow of blood through the inflow side of the valve 100.

As stated previously, asymmetry in the leaflet window maybe be affected by various geometries. In the embodiment of FIG. 5C, the leaflet window 137 defines the shape of three sides of an asymmetric trapezoid. The embodiment of FIG. 2A is an example of an asymmetric parabola. The embodiment of FIG. 4 is an example of a symmetric parabola with asymmetric commissure post attachment which may also result in an asymmetric coaptation region which may affect leaflet dynamics in a preferential way to produce a spiral downstream flow.

The embodiments discussed above are examples of three different leaflet and leaflet window geometries that are suitable for the particular purpose of asymmetric opening of the valve that produces an asymmetric flow condition. It is understood that other leaflet and leaflet window geometries may also be suitable for the particular purpose and are not limited thereto. In accordance with an embodiment, the leaflet first side region of the leaflet has a different geometry relative to the leaflet second side region. The different geometric characteristics of the leaflet first side region and the leaflet second side region may be affected by any suitable means, such as, but not limited to different attachment line lengths, different shape of the attachment line, and different surface areas of the two side regions.

It is understood that the leaflet frame may comprise any number of leaflet windows, and thus leaflets, suitable for a particular purpose, in accordance with embodiments. Leaflet frames comprising one, two, three or more leaflet windows and corresponding leaflets are anticipated.

Although embodiments provided above comprise a leaflet frame that supports the leaflets, it is understood and appreciated that the leaflets may not necessarily be supported by a frame. In accordance with an embodiment, the leaflets may be supported by the wall of a solid-walled conduit without a frame that defines leaflet windows and commissure posts. In other embodiments, the leaflets may be constructed as in the tissue valve art that are formed into the desired shape without a frame.

In another embodiment of a prosthetic valve including a plurality of leaflets, each leaflet includes a leaflet first side and a leaflet second side opposite from the leaflet first side. Each leaflet first side is coupled with the leaflet second side of an adjacent leaflet at a commissure. The plurality of leaflets defines a lumen, also referred to as a lumen, when the leaflets are in an open position. Each of the leaflet second sides extend further into the lumen than each of the leaflet first sides.

In another embodiment, a prosthetic valve comprises a plurality of leaflets. Each leaflet includes a leaflet first side region and a leaflet second side region opposite from the leaflet first side region. Each leaflet defines a leaflet base and a leaflet free edge opposite from the leaflet base. Each leaflet first side region is coupled with the leaflet second side region of an adjacent leaflet at a commissure. The leaflet base of the plurality of leaflets defines a lumen. The leaflet second side regions extend further into the lumen than the leaflet first side region when the leaflets are in the fully open position.

In another embodiment, a prosthetic valve comprises a plurality of leaflets. Each leaflet includes a leaflet first side region and a leaflet second side region opposite from the leaflet first side region. At least a first portion of the leaflet first side region has a first surface area and the leaflet second side region has a second surface area wherein the first surface area is greater than the second surface area. In operation, each leaflet opens asymmetrically. In one embodiment, the first surface area may be 1.2 times greater than the second surface area. This paragraph is a place holder for a discussion of surface area.

In another embodiment, a prosthetic valve comprises a plurality of leaflets. Each leaflet includes a leaflet first side region and a leaflet second side region opposite from the leaflet first side region. The leaflet first side region has a first bending characteristic and the leaflet second side region has a second bending characteristic. The first bending characteristic is operable to open asymmetrically relative to the second bending characteristic. In operation, each leaflet opens asymmetrically. This bending characteristic is affected by, such as, the geometry or length of the attachment line of a leaflet first side region as compared with a leaflet second side region.

In another embodiment, a prosthetic valve comprises a plurality of leaflets. Each leaflet includes a leaflet first side region and a leaflet second side region opposite from the leaflet first side region. The leaflet first side region being less resistant to moving compared with the leaflet second side region. In operation, each leaflet opens asymmetrically. This resistant to moving characteristic is affected by, such as, the geometry or length of the attachment line of a leaflet first side region as compared with a leaflet second side region.

In another embodiment, a prosthetic valve comprises a plurality of leaflets. Each leaflet includes a leaflet first side region and a leaflet second side region opposite from the leaflet first side region. The leaflet first side region being faster to open compared with the leaflet second side region. In operation, each leaflet opens asymmetrically. This speed to open characteristic is affected by, such as, the geometry or length of the attachment line of a leaflet first side region as compared with a leaflet second side region.

In another embodiment, a prosthetic valve comprises a plurality of leaflets. Each leaflet includes a leaflet first side region and a leaflet second side region opposite from the leaflet first side region. Each leaflet defines a leaflet base and a leaflet free edge opposite from the leaflet base. Each leaflet first side region is coupled with the leaflet second side region of an adjacent leaflet at a commissure. The leaflet base of the plurality of leaflets defines a lumen. At least one of the leaflet second side regions extends further into the lumen than the leaflet first side region when the leaflets are in the fully open position. This extension into the lumen characteristic is affected by, such as, the geometry or length of the attachment line of a leaflet first side region as compared with a leaflet second side region.

### Leaflet Materials

In various examples, the leaflet 140 is formed of a biocompatible, synthetic material (e.g., including ePTFE and ePTFE composites, or other materials as desired). In other examples, the leaflet 140 is formed of a natural material, such as repurposed tissue, including bovine tissue, porcine tissue, or the like.

Some examples of suitable leaflet materials may be found in U.S. Pat. No. 8,961,599 to Bruchman et al. (“Durable High Strength Polymer Composite Suitable for Implant and Articles Produced Therefrom”); U.S. Pat. No. 8,945,212 to Bruchman et al. (“Durable Multi-Layer High Strength Polymer Composite Suitable for Implant and Articles Produced Therefrom”); U.S. Pat. No. 9,554,900 to Bruchman et al. (“Durable High Strength Polymer Composites Suitable for Implant and Articles Produced Therefrom”); and U.S. Pat. App. Pub. 2015/0224231 to Bruchman et al. (“Coherent Single Layer High Strength Synthetic Polymer Composites for Prosthetic Valves”).

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material.

In accordance with embodiments herein, the leaflet 140 comprises a composite material having at least one porous synthetic polymer membrane layer having a plurality of pores and/or spaces and an elastomer and/or an elastomeric material and/or a non-elastomeric material filling the pores and/or spaces of the at least one synthetic polymer membrane layer. In accordance with other examples, the leaflet 140 further comprises a layer of an elastomer and/or an elastomeric material and/or a non-elastomeric material on the composite material. In accordance with examples, the composite material comprises porous synthetic polymer membrane by weight in a range of 10% to 90%.

An example of a porous synthetic polymer membrane includes expanded fluoropolymer membrane having a node and fibril structure defining the pores and/or spaces. In some examples, the expanded fluoropolymer membrane is expanded polytetrafluoroethylene (ePTFE) membrane. Another example of porous synthetic polymer membrane includes microporous polyethylene membrane.

Examples of an elastomer and/or an elastomeric material and/or a non-elastomeric material include, but are not limited to, copolymers of tetrafluoroethylene and perfluoromethyl vinyl ether (TFE/PMVE copolymer), (per)fluoroalkylvinylethers (PAVE), urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing. In some examples, the TFE/PMVE copolymer is an elastomer comprising between and including 60 and 20 weight percent tetrafluoroethylene and respectively between and including 40 and 80 weight percent perfluoromethyl vinyl ether. In some examples, the TFE/PMVE copolymer is an elastomeric material comprising between and including 67 and 61 weight percent tetrafluoroethylene and respectively between and including 33 and 39 weight percent perfluoromethyl vinyl ether. In some examples, the TFE/PMVE copolymer is a non-elastomeric material comprising between and including 73 and 68 weight percent tetrafluoroethylene and respectively between and including 27 and 32 weight percent perfluoromethyl vinyl ether. The TFE and PMVE components of the TFE-PMVE copolymer are presented in wt %. For reference, the wt % of PMVE of 40, 33-39, and 27-32 corresponds to a mol % of 29, 23-28, and 18-22, respectively.

In some examples, the TFE-PMVE copolymer exhibits elastomer, elastomeric, and/or non-elastomeric properties.

In some examples, the composite material further comprises a layer or coating of TFE-PMVE copolymer comprising from 73 to 68 weight percent tetrafluoroethylene and respectively from 27 to 32 weight percent perfluoromethyl vinyl ether.

In some examples, the leaflet 140 is an expanded polytetrafluoroethylene (ePTFE) membrane having been imbibed with TFE-PMVE copolymer comprising from 60 to about 20 weight percent tetrafluoroethylene and respectively from 40 to about 80 weight percent perfluoromethyl vinyl ether, the leaflet 1230 further including a coating of TFE-PMVE copolymer comprising from 73 to 68 weight percent tetrafluoroethylene and respectively 27 to 32 weight percent perfluoromethyl vinyl ether on the blood-contacting surfaces.

As discussed above, the elastomer and/or an elastomeric material and/or a non-elastomeric material may be combined with the expanded fluoropolymer membrane such that the elastomer and/or the elastomeric material and/or the non-elastomeric material occupies substantially all of the void space or pores within the expanded fluoropolymer membrane.

Although some examples of suitable leaflet materials have been provided, the foregoing examples are not meant to be read in a limiting sense, and additional or alternative materials are contemplated.

It will be apparent to those skilled in the art that various modifications and variations can be made in the present embodiments without departing from the spirit or scope of the embodiments. Thus, it is intended that the present embodiments cover the modifications and variations of this invention provided they come within the scope of the appended claims and their equivalents.

What is claimed:

1. A prosthetic valve comprising:
a leaflet frame defining a plurality of leaflet windows each defining an attachment zone, each attachment zone defining three sides of an asymmetric acute trapezoid; and
a leaflet extending from each of the leaflet windows, the leaflet defining a leaflet base and a free edge opposite the leaflet base and two leaflet sides having dissimilar length, the leaflet base and two leaflet sides being coupled to the respective attachment zone,
wherein adjacent leaflet free edges are configured to coapt in a progressive manor when closing and disengage in a progressive manor when opening.

2. A prosthetic valve comprising:
a tubular support structure defining a lumen; and
a plurality of leaflets coupled to the support structure, each leaflet defining a leaflet base and a free edge opposite the leaflet base and two leaflet sides having dissimilar length, the leaflet base and two leaflet sides being coupled to the support structure and in cooperative engagement with respective leaflets such that adjacent free edges coapt when in a closed position so as to occlude the lumen,
wherein adjacent leaflet free edges are configured to coapt in a progressive manor from adjacent the support structure to a point of convergence when closing and disengage in a progressive manor from the point of convergence to the support structure when opening.

3. A prosthetic valve comprising:
a leaflet frame defining a plurality of leaflet windows each defining an attachment zone in the shape of an asymmetric parabola; and
a leaflet extending from each of the leaflet windows, the leaflet defining two leaflet sides having dissimilar length, the two leaflet sides being coupled to the attachment zone,
wherein adjacent leaflet free edges are configured to coapt in a progressive manor when closing and disengage in a progressive manor when opening.

4. A prosthetic valve comprising:
a tubular support structure defining a lumen; and
a plurality of leaflets coupled to the support structure, each leaflet defining two leaflet sides having dissimilar length, the leaflet base and two leaflet sides in the shape of an asymmetric parabola being coupled to the support structure and in cooperative engagement with respective leaflets such that adjacent free edges coapt when in a closed position so as to occlude the lumen,
wherein adjacent leaflet free edges are configured to coapt in a progressive manor from adjacent the support structure to a point of convergence when closing and disengage in a progressive manor from the point of convergence to the support structure when opening.

5. A prosthetic valve comprising:
a plurality of leaflets, each leaflet defines a leaflet first side region and a leaflet second side region opposite from the leaflet first side region, each leaflet defining a leaflet base and a leaflet free edge opposite from the leaflet base, each leaflet first side region is coupled with the leaflet second side region of an adjacent leaflet at a commissure, the leaflet base of each of the plurality of leaflets together define a lumen, at least one of the leaflet second side regions extend further into the lumen than the leaflet first side region when the leaflets are in a fully open position.

6. A prosthetic valve comprising:
a leaflet frame defining a plurality of leaflet windows each defining an attachment zone in the shape of a symmetric parabola and a first commissure post and second commissure post extending therefrom; and
a leaflet extending from each of the leaflet windows, each leaflet having a first tab attachment line operable to couple with the first commissure post, and a second tab attachment line operable to couple with the second commissure post, the second tab attachment line has a greater angle relative to a valve longitudinal axis as compared with the first tab attachment line having a smaller angle.

7. A prosthetic valve comprising:
a leaflet frame defining a plurality of leaflet windows each defining an attachment zone in the shape of a symmetric parabola and a first commissure post and second commissure post extending therefrom; and
a leaflet extending from each of the leaflet windows, each leaflet having a first tab attachment line operable to couple with the first commissure post, and a second tab attachment line operable to couple with the second commissure post, the first tab attachment line has a greater length relative to the second tab attachment line.

8. The prosthetic valve of any one of claims 1 through 7, wherein when in an open position, the leaflet second side region contributes to a smaller geometric orifice area as compared with the leaflet first side region.

9. The prosthetic valve of claim 8, wherein the leaflet first side region opens further than the leaflet second side region during forward flow.

10. The prosthetic valve of claim 8 wherein when in the open position, the leaflet second side region has between a 10 to 70 percent smaller geometric orifice area as compared with the leaflet first side region.

11. The prosthetic valve of any one of claims 1 through 7, wherein the leaflet first side region has a first surface area and the leaflet second side region has a second surface area, the first surface area being greater than the second surface area.

12. The prosthetic valve of any one of claims 1 through 7, wherein the leaflet comprises at least one layer of a composite material.

13. The prosthetic valve of any one of claims 1 through 7 wherein the attachment line of the leaflet first side region is longer than the attachment line of the leaflet second side region.

14. The prosthetic valve of any one of claims 1 through 7, wherein the leaflet comprises a polymeric material.

15. The prosthetic valve of any one of claims 1 through 7, wherein each of the leaflet windows includes a leaflet window first side, a leaflet window second side opposite the leaflet window first side, and a leaflet window base therebetween, wherein a leaflet window side of one leaflet window is interconnected with a leaflet window side of an adjacent leaflet window by the leaflet window base,
wherein the leaflet first side region being coupled to the leaflet window first side, the leaflet second side region being coupled to the leaflet window second side and the leaflet base being coupled to the leaflet window base.

16. The prosthetic valve of any one of claims 3 and 4, wherein each of the leaflet windows includes a leaflet window first side and a leaflet window second side opposite the leaflet window first side, wherein a leaflet window side of one leaflet window is interconnected with a leaflet window side of an adjacent leaflet window,
wherein the leaflet first side region being coupled to the leaflet window first side, and the leaflet second side region being coupled to the leaflet window second side.

17. The prosthetic valve of any one of claims 15 and 16, wherein two adjacent leaflet window first side and leaflet window second side terminates at a commissure post.

18. The prosthetic valve of claim 17, each leaflet including a free edge, a base opposite from the free edge and coupled to the leaflet window base, wherein the leaflet reinforcing member extends to the free edge of the leaflet.

19. The prosthetic valve of claim 17, further comprising a vertical element extending from each of the commissure posts.

20. The prosthetic valve of any one of claims 1 through 7, wherein the leaflet comprises a polymeric material.

21. The prosthetic valve of claim 20, wherein the leaflet comprises a laminate.

22. The prosthetic valve of claim 21, wherein the laminate has more than one layer of a fluoropolymer membrane.

23. The prosthetic valve of claim 20, wherein the leaflet comprises at least one fluoropolymer membrane having a plurality of pores and an elastomer present in substantially all of the pores of the at least one fluoropolymer membrane.

24. The prosthetic valve of claim 23, wherein the film comprises less than 80% fluoropolymer membrane by weight.

25. The prosthetic valve of claim 23, wherein the elastomer comprises (per)fluoroalkylvinylethers (PAVE).

26. The prosthetic valve of claim 23, wherein the elastomer comprises a copolymer of tetrafluoroethylene and perfluoromethyl vinyl ether.

27. The prosthetic valve of claim 23, wherein the fluoropolymer membrane comprises ePTFE.

28. A method of treating a failing or dysfunctional native heart valve with a prosthetic valve, the method comprises:
replacing the native valve with a prosthetic valve in accordance with any one of claims 1 through 27.
